(19) United States
(12) Patent Application Publication
(10) Pub. No.: US 2004/0247597 A1
(43) Pub. Date: Dec. 9, 2004

(54) Method of treating atherosclerosis and other inflammatory diseases

(76) Inventors: Peter Carmeliet, Landen (BE); Daniel J. Hicklin, Upper Montclair, NJ (US); Fang Liao, New York City, NY (US); Yan Wu, Flemington, NJ (US); Aernout Luttun, Zwevezele (BE)

Correspondence Address: KENYON & KENYON, ONE BROADWAY, NEW YORK, NY, 10004, US

(21) Appl. No.: 10/482,269

(22) PCT Filed: Jun. 20, 2002
(86) PCT No.: PCT/US02/19505

Related U.S. Application Data
(60) Provisional application No. 60/299,610, filed on Jun. 20, 2001.
(60) Provisional application No. 60/355,318, filed on Feb. 8, 2002.

(51) Int. Cl. A61K 39/395; A61K 38/17

(52) U.S. Cl. 424/145.1; 514/12

(57) ABSTRACT

The present invention is directed to methods utilizing vascular endothelial growth factor receptor (VEGFR) antagonists to treat atherosclerosis and other inflammatory diseases.

### FIELD OF THE INVENTION

The present invention is directed to methods utilizing vascular endothelial growth factor receptor (VEGFR) antagonists to treat atherosclerosis and other inflammatory diseases.

### BACKGROUND OF THE INVENTION

Atherosclerosis and its complications are a major cause of death in the United States. Atherosclerosis involves the progressive narrowing and hardening of the arteries, which over time leads to ischemia or infarction in different tissues or organs. The most common single cause of death, which accounts for one third of all deaths, is atherosclerotic heart disease involving the coronary arteries, causing angina and heart attacks. Atherosclerotic interference with blood supply to the brain, causing strokes, is the third most common cause of death after cancer. Atherosclerosis also causes a great deal of serious illness by reducing the blood flow in other major arteries, such as those to the kidneys, the legs and the intestines, thus causing peripheral artery disease.

To some degree, atherosclerosis is known to occur with aging, but other risk factors that accelerate this process have been identified, including high cholesterol (hypercholesterolemia), high blood pressure, smoking, and family history for atherosclerotic disease. The condition also may be secondary to another disorder, such as Systemic Lupus Erythematosus (SLE), hypothyroidism, Nephrotic Syndrome, Cushing's Syndrome, Diabetes Mellitus, obesity, alcoholism, corticosteroid therapy or estrogen therapy. Although less carefully studied, other heart disease risk factors, such as high levels of homocysteine and lack of exercise, also play important roles. In addition, elevated levels of C-reactive protein may predict a high risk of future peripheral artery disease.

Various atherosclerosis treatments currently exist. Medication is not a satisfactory treatment because much of the damage to the artery walls has already been done. Anticoagulant drugs have been used to try to minimize secondary clotting and embolus formation, but have little or no effect on the progress of the disease. Vasodilator drugs are used to provide symptom relief, but are of no curative value.

Surgical treatment is also available for certain high-risk situations. Balloon angioplasty can open up narrowed vessels and promote an unproved blood supply. Recently, a newer technique of inserting a metallic stenting element has been used to permanently maintain the walls of the vessel treated in its extended opened state. Vascular stents are tiny mesh tubes made of stainless steel or other metals and are used by heart surgeons to prop open the weak inner walls of diseased arteries. They are often used in conjunction with balloon angioplasty to prevent re-stenosis after the clogged arteries are treated. The blood supply to the heart muscle can also be restored through a vein graft bypass. Large atheromatous and calcified arterial obstructions can be removed by endarterectomy, and woven plastic tube grafts can replace entire segments of diseased peripheral vessels.

With regard to reduction of hypercholesterolemia, one of the risk factors for atherosclerosis, in some instances this can be achieved by modification of the diet and/or use of drugs, thereby minimizing the risk of fatality of the disease. Consumption of dietary plant fiber and other effective components of foods have achieved reduction of serum cholesterol in humans.

Although atherosclerosis is the most common cause of the narrowing of a limb artery, other disorders may also be responsible. Blood clots (thrombi) can form in the heart or in the aorta or other large arteries. Clots can break loose and travel in the bloodstream (embolize) until they lodge in a smaller artery. Cholesterol emboli, which are less urgent, occur when tiny particles of cholesterol break off from plaques and block small arteries. A different condition, popliteal artery entrapment, occurs primarily in healthy young individuals when an enlargement of the calf muscles puts excessive pressure on the artery below it. Popliteal artery entrapment occurs predominantly in men; in contrast, Raynaud's phenomenon is more common in women. In this condition, cold temperatures cause temporary arterial spasms that produce painful blue or white fingers or toes. Finally, inflammation of an artery (arteritis) can also limit blood flow to a limb.

As a systemic disease, atherosclerosis can affect any artery in the body. Even so, the damage is remarkably focal, with plaques damaging one particular, small stretch of an artery, while sparing adjacent segments. For example, the plaques of peripheral artery disease are much more common in the legs than the arms. And in the abdomen and legs, the blockages tend to occur in specific locations, such as the aorta and the iliac, femoral, popliteal, and tibial arteries. The points of greatest risk are the places where the arteries branch into smaller vessels.

Atherosclerosis begins when cholesterol passes from the blood across the endothelium into an artery's middle layer. Cholesterol is the culprit, but not all forms are culpable; in fact, the damage is initiated by oxidized LDL cholesterol, the “bad” cholesterol in the blood that is itself modified into a toxic form by the action of oxygen free radicals. Oxidized cholesterol triggers a sequence of events in the artery wall, ultimately leading to atherosclerosis. Macrophages migrate into the artery, where they engulf the oxidized cholesterol. When macrophages engulf bacteria and viruses, they kill the microbes, but in the case of cholesterol, the reverse is true. After engulfing the oxidized cholesterol, the macrophages enlarge into foam cells, then die, releasing their contents and stimulating even more inflammation and creating a fatty streak. In response, the smooth muscle cells enlarge. The result is a plaque with an inflammatory center and a hard shell, which gradually encroaches on the artery's channel, blocking the flow of blood. If the plaque ruptures, exposing the inflammatory core to the bloodstream, platelets adhere to the plaque, where they initiate thrombosis, or clot formation. The clot, not the plaque, is usually responsible for the complete blockage of an artery.

Angiogenic factors may stimulate the growth and the vulnerability to rupture of plaques via intraplaque neovascularization. The clinical importance of plaque neovascularization is underscored by the higher prevalence of neovascularization in lesions from patients with unstable angina as well as in lesions with rupture and mural hemorrhage. Previous studies have identified many peptide growth factors, including platelet-derived growth factor (PDGF), acid and basic fibroblast growth factor (aFGF and bFGF, respectively), angiotensin II, and transforming growth factor-β (TGF-β), and inflammatory cytokines, such as tumor necrosis factor-α (TNF-α) and interleukin-1β, as being responsible for the activation of vascular cells. Expression of FGF's in human atherosclerotic plaques increased smooth muscle cell accumulation and lesion instability, while transgenic expression of aFGF exaggerated neointimal thickening in animal models.

In addition, evidence indicates that vascular endothelial growth factor (VEGF), the biological response of which is mediated through its high affinity VEGF receptors (VEGFRs), plays an important role in the development of atherosclerosis. High expression of the angiogenic factor VEGF has been detected in total occlusive atherosclerotic lesions with extensive neovascularization. Furthermore, similar to aFGF, administration of VEGF also exaggerated neointimal thickening in animal models. VEGF and the VEGF homologue placenta growth factor (P1GF) are also both present in infiltrating plaques, while VEGF can be released within plaques upon aggregation. Moulton et al., Circulation, 99: 1726-32 (1999), demonstrated that prolonged treatment with two different angiogenesis inhibitors reduced intimal neovascularization and plaque growth in the apolipoprotein E deficient (apoE−/−) mouse model, while administration of VEGF promoted atherosclerotic plaque growth.

In addition to their presence in infiltrating plaques, VEGF and P1GF are also both present in inflammatory cells. These growth factors may play a role in other inflammatory diseases, as both VEGF and P1GF are known chemoattractants for inflammatory cells, i.e. monocytes/macrophages, and the latter cells carry their receptor, VEGFR-1. Inflammatory diseases include chronic inflammatory diseases, which are characterized by a progressive and sustained anti-self response, i.e., an autoimmune response, typically leading to development of tissue inflammation and, in severe cases, destruction of the inflamed tissue. The inflammatory cytokines IL-1 and TNF-α, as well as macrophages, monocytes and granulocytes, play an important role in a number of these inflammatory diseases. See, e.g., Dinarello et al., Curr. Opin. Immunol., 3: 941-48 (1991).

Present treatment regimens of inflammatory diseases involve the use of glucocorticoids, non-steroidal anti-inflammatory drugs (NSAIDs) and, for RA, disease-modifying anti-rheumatic drugs (DMARDs), or combinations of these. Negative side effects and poor efficacy have been serious drawbacks for these therapeutic agents. New anti-inflammatory drugs inhibiting TNF-α function (etanercept, infliximab) have recently been approved for clinical use in RA.

### BRIEF DESCRIPTION OF THE FIGURES

FIG. 1 graphically demonstrates the binding characteristics of a VEGFR antagonist, the monoclonal antibody MF-1, to VEGFR-1.

FIG. 2 graphically demonstrates the blocking characteristics of a VEGFR antagonist, the monoclonal antibody MF-1. FIG. 2A shows MF-1 blocking VEGFR-1 binding to P1GF, and FIG. 2B shows MF-1 blocking VEGFR-1 binding to VEGF.

### BRIEF SUMMARY OF THE INVENTION

The present invention relates to methods of treating atherosclerosis and other inflammatory diseases by administering a VEGFR antagonist that inhibits binding of VEGF and/or P1GF to the receptor. In one embodiment, the antagonist is an antibody that binds VEGFR-1, such that VEGF and P1GF are inhibited from binding to the VEGFR-1 receptor.

### DETAILED DESCRIPTION OF THE INVENTION

Angiogenic factors, such as VEGF and P1GF, may promote plaque progression via several mechanisms acting at different stages of atherosclerosis. First, both growth factors are known chemoattractants for inflammatory cells and recruit these cells in atherosclerotic lesions, thereby mediating plaque growth in initial stages and possibly contributing to plaque destabilization at later stages. Second, plaque neovascularization induced by locally secreted angiogenic factors may be a prerequisite for growth of the lesion beyond a certain size, as diffusion of oxygen from the vessel lumen is insufficient to meet the metabolic demands of the plaque. Third, VEGF and P1GF, which stimulate fibrin formation via expression of tissue factor, initiating coagulation, may affect plaque growth by increasing vascular permeability and extravasation of plasma proteins, including fibrinogen, fibronectin and others proteins that constitute a scaffold for migrating wound cells. Finally, VEGF and P1GF stimulate smooth muscle cell (SMC) proliferation, as shown by findings that exogenous VEGF induced prominent intimal thickening in rabbit carotid arteries and exacerbated neointimal thickening after vascular injury in dogs.

As VEGF is highly expressed in atherosclerotic lesions, P1GF may also play a role in the progression of atherosclerosis by modulating the effect of VEGF on plaque neovascularization, macrophage recruitment and SMC proliferation. In addition, P1GF may have a direct effect on macrophage recruitment and fatty streak formation, as the latter cells express its receptor. The possible involvement of the angiogenic factors VEGF and P1GF in the development of atherosclerosis has important clinical implications, as counteracting these growth factors might constitute a possible approach to induce lesion regression. Because the receptors for VEGF do not interfere with physiological functions, suppression of the receptors for VEGF is a better target for therapy than suppression of VEGF itself, as the latter may cause neurodegeneration.

Angiogenic factors like VEGF and P1GF may also perpetuate and exacerbate inflammatory diseases by a variety of different routes. Rheumatoid arthritis (RA), which is a prime example of such inflammatory diseases, is illustrated in this section. Angiogenesis in the synovium is one of the early events in the pathogenesis of RA and is important in the progression of the disease, as infiltration of inflammatory cells and pannus growth is largely dependent on the presence of new vessels. VEGF and P1GF may mediate several pathological features of RA, since many cell types present in the arthritic joint may carry one or more receptor-types for these angiogenic factors. While vascular cells (endothelial cells and smooth muscle cells) have both VEGFRs, cells of the monocyte-macrophage lineage (macrophages, monocytes and osteoclasts) only express one VEGFR, which is a receptor for both VEGF and P1GF. The expression pattern of this VEGFR indicates that VEGF or P1GF may not only mediate synovial neovascularization, but also infiltration of inflammatory cells and bone destruction. In addition, since type A synovial cells derive from the monocyte lineage, these cells may also express the VEGFR and proliferate in response to VEGF or P1GF.

VEGF is a key regulator of vasculogenesis, which is the de novo development of new blood vessels from the differentiation of endothelial precursors (angioblasts) in situ, during embryonic development and angiogenic processes during adult life, such as wound healing, diabetic retinopathy, RA, psoriasis, inflammatory disorders, tumor growth and metastasis. VEGF, which is a homodimeric glycoprotein consisting of two 23 kD subunits, is a strong inducer of vascular permeability, stimulator of endothelial cell migration and proliferation, and an important survival factor for newly formed blood vessels. Four different monomeric isoforms of VEGF exist, resulting from alternative splicing of mRNA. These include two membrane bound forms (VEGF₂₀₆ and VEGF₁₈₉) and two soluble forms (VEGF₁₆₅ and VEGF₁₂₁). In all human tissues except placenta, VEGF₁₆₅ is the most abundant isoform.

The biological response of VEGF is mediated through its high affinity VEGFRs, which typically are class III receptor-type tyrosine kinases characterized by having several, generally 5 or 7, immunoglobulin-like loops in their amino-terminal extracellular receptor ligand-binding domains. The other two regions include a transmembrane region and a carboxy-terminal intracellular catalytic domain interrupted by an insertion of hydrophilic interkinase sequences of variable lengths, called the kinase insert domain. VEGFRs include fms-like tyrosine kinase receptor (fit-1) or VEGFR-1, sequenced by Shibuya et al., Oncogene, 5: 519-524 (1990), kinase insert domain-containing receptor/fetal liver kinase (KDR/flk-1) or VEGFR-2, described in WO 92/14248, filed Feb. 20, 1992, and Terman et al., Oncogene, 6: 1677-1683 (1991) and sequenced by Matthews et al., Proc. Natl. Acad. Sci. USA, 88: 9026-9030 (1991), although other receptors, such as neuropilin-1 and -2, can also bind VEGF. Another tyrosine kinase receptor, VEGFR-3 (flt-4) binds the VEGF homologues VEGF-C and VEGF-D and is more important in the development of lymphatic vessels. Ligands for VEGFR include VEGF and its homologues P1GF, VEGF-B, VEGF-C, VEGF-D, and VEGF-E.

It is generally believed that VEGFR-2 is the main VEGF signal transducer that results in endothelial cell proliferation, migration, differentiation, tube formation, increase of vascular permeability, and maintenance of vascular integrity. VEGFR-1 possesses a much weaker kinase activity, and is unable to generate a mitogenic response when stimulated by VEGF—although it binds to VEGF with an affinity that is approximately 10-fold higher than VEGFR-2. VEGFR-1 is also been implicated in VEGF induced migration of monocytes/macrophage and production of tissue factor.

The VEGF homologue P1GF is also a natural specific ligand for VEGFR-1. P1GF, a dimeric secreted factor, is produced in large amounts by villous cytotrophoblast, sincytiotrophoblast and extravillous trophoblast and has close amino acid homology to VEGF. Three isoforms exist in humans, P1GF-1, P1GF-2, and P1GF-3. Studies with P1GF-deficient mice demonstrate that this growth factor is not involved in angiogenesis per se, instead it specifically modulates the angiogenic and permeability effects of VEGF during pathological situations.

The present invention provides methods utilizing VEGFR antagonists to treat atherosclerosis and other inflammatory diseases. The VEGFR antagonist of the present invention can be an antibody, a ligand, a peptide, a DNA, a small molecule, or any other suitable antagonist. In order to be useful, the antibody, ligand, peptide, DNA, or small molecule must be sufficient to block binding to the receptor by at least one of the VEGFR ligands and/or prevent activation of the VEGF sub family of receptors, including preventing activation resulting from higher levels of ligand, VEGFR gene amplification, increased VEGFR transcription or mRNA translation, stability of the receptor, or mutations that cause unregulated receptor signaling.

In the context of the present invention, by prevention of activation of the VEGFR is meant any decrease in the activation of the VEGFR. That is, the prevention of activation need not completely halt activation of the VEGFR. Prevention of activation of the VEGF sub family of receptors can prevent any of the following activities: receptor dimerization, autophosphorylation of VEGFR, activation of the receptor's internal, cytoplasmic tyrosine kinase domain, and initiation of multiple signal transduction pathways involved in regulation of vasculogenesis and angiogenesis. Such signal transduction pathways include, e.g., the phospholipase Cγ (PLCγ) pathway or the phosphatidylinositol 3′ kinase (PI3-K)/Akt and mitogen activated protein kinase (MAPK) pathway. See, e.g., Larrivée et al., Int'l J. Mol. Med., 5: 447-56 (2000).

A preferred VEGFR antagonist is one that prevents either VEGF or P1GF from binding to the VEGFR. This can be accomplished by any suitable means. For example, direct binding of the VEGFR antagonist to the VEGFR or direct binding of the VEGFR antagonist to VEGF or P1GF can be utilized to prevent VEGF or P1GF from binding to the VEGFR. It should be appreciated that when the VEGFR antagonist specifically binds VEGFR, the antagonist can bind externally to the extracellular portion of VEGFR, which may or may not inhibit binding of the ligand, or internally to the tyrosine kinase domain.

Examples of VEGFR antagonists that bind VEGFR include, without limitation, biological molecules, such as receptor ribozymes and antibodies (or functional equivalents thereof) specific for VEGFR, and synthetic kinase inhibitors that act directly on the cytoplasmic domain of VEGFR, such as small molecules. Preferably, the VEGFR antagonist of the present invention is a biological molecule and more preferably, an antibody, or functional equivalent thereof, specific for VEGFR, details of which are described in more detail below. Alternatively, the VEGFR antagonist of the present invention is a small molecule kinase inhibitor; again, details of which are described in more detail below.

In one embodiment of the present invention in which the VEGFR antagonist is an antibody, the antibody can be a monoclonal antibody, a fragment of an antibody, a derivative of an antibody, a chimerized antibody (constant region from an antibody of one species and variable region from an antibody of another species), a humanized antibody (constant region from an antibody from a human and complementarity-determining regions (CDRs) and framework regions of an antibody from another species), or a fully human antibody. The antibody can also be a single chain antibody (scFv) or a synthetic homolog of the antibody. As used herein, unless otherwise indicated or clear from the context, antibody domains, regions and fragments are accorded standard definitions as are well known in the art. See, e.g., Abbas et al., Cellular and Molecular Immunology, W. B. Saunders Company, Philadelphia, Pa. (1991). The VEGFR antagonist antibodies of the subject invention are preferably monoclonal.

The VEGFR antagonist can also be a fragment of an antibody. The fragment can be produced by cleaving a whole antibody, or by expressing DNA that encodes the fragment. Fragments of antibodies can be prepared by methods described by Lamoyi et al., J. Immunol. Methods, 56: 235-243 (1983) and by Parham, J. Immunol. 131: 2895-2902 (1983). Fragments of antibodies useful in the invention have the same binding characteristics as, or that have binding characteristics comparable to, those of the whole antibody. Such fragments can contain one or both Fab fragments or the F(ab′)₂ fragment. Such fragments can also contain single-chain fragment variable region antibodies, i.e. scFv, dibodies, or other antibody fragments. Preferably the antibody fragments contain all six complementarity-determining regions of the whole antibody, although fragments containing fewer than all of such regions, such as three, four or five CDRs, can also be functional. If the antibody fragment is too short to be immunogenic, it can be conjugated to a carrier molecule. Some suitable carrier molecules include keyhole limpet hemocyanin and bovine serum albumen. Conjugation can be carried out by methods known in the art.

Antibodies that are VEGFR antagonists of the invention include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al., J. Mol. Bio., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Bio., 226: 889-896 (1992)). Phage display vectors containing heavy and light chain variable region genes are propagated in mutator strains of E. coli (see, e.g., Low et al., J. Mol. Bio., 250: 359-368 (1996)). These methods of mutagenesis are illustrative of the many methods known to one of skill in the art.

In one preferred embodiment, the VEGFR antagonist binds specifically to VEGFR-1. Particularly preferred are antigen-binding proteins that bind to the extracellular domain of VEGFR-1 and block binding by one of its ligands and/or neutralize ligand-induced activation of VEGFR-1. For example, MAb 6.12 is a scFv that binds to soluble and cell surface-expressed VEGFR-1. ScFv 6.12 has the V_{L} and V_{H} domains of mouse monoclonal antibody MAb 6.12. A hybridoma cell line producing MAb 6.12 has been deposited as ATCC number PTA-3344. The deposit was made under the provisions of the Budapest Treaty on the International Recognition of the Deposit of Microorganisms for the Purposes of Patent Procedure and the regulations thereunder (Budapest Treaty). This assures maintenance of a viable culture for 30 years from date of deposit. The organisms will be made available by ATCC under the terms of the Budapest Treaty, and subject to an agreement between Applicants and ATCC, which assures unrestricted availability upon issuance of the pertinent U.S. patent. Availability of the deposited strains is not to be construed as a license to practice the invention in contravention of the rights granted under the authority of any government in accordance with its patent laws.

There also exist various hybridomas that produce VEGFR-2 antibodies. For example, a hybridoma cell line producing rat anti-mouse VEGFR-2 monoclonal antibody (DC101) was deposited as ATCC HB 11534; a hybridoma cell line (M25.18A1) producing mouse anti-mouse VEGFR-2 monoclonal antibody MAb 25 was deposited as ATCC HB 12152; a hybridoma cell line (M73.24) producing mouse anti-mouse VEGFR-2 monoclonal antibody MAb 73 was deposited as ATCC HB 12153.

In addition, there are various hybridomas that produce anti-VEGFR-1 antibodies include, but not limited to, hybridomas KM1730 (deposited as FERM BP-5697), KM1731 (deposited as FERM BP-5718), KM1732 (deposited as FERM BP-5698), KM1748 (deposited as FERM BP-5699), KM1750 (deposited as FERM BP-5700) disclosed in WO 98/22616, WO 99/59636, Australian accepted application no. AU 1998 50666 B2, and Canadian application no. CA 2328893.

Many other VEGFR antagonists are known in the art. Some examples of VEGFR antagonists are described in U.S. application Ser. Nos. 07/813,593; 07/906,397; 07/946,507; 07/977,451; 08/055,269; 08/252,517; 08/601,891; 09/021,324; 09/208,786; and 09/919,408 (all to Lemischka et al.); U.S. Pat. No. 5,840,301 (Rockwell et al.); U.S. application Ser. Nos. 08/706,804; 08/866,969; 08/967,113; 09/047,807; 09/401,163; and 09/798,689 (all to Rockwell et al.); U.S. application Ser. No. 09/540,770 (Witte et al.); and PCT/US01/06966 (Liao et al.). U.S. Pat. No. 5,861,301 (Terman et al.), Terman et al. Oncogene 6: 1677-1683 (September 1991), WO 94/10202 (Ferrara et al.), and WO 95/21865 (Ludwig) disclose VEGFR antagonists and, specifically, anti-VEGFR-2 antibodies. In addition, PCT/US95/01678 (Kyowa Hakko) describes anti-VEGFR-2 antibodies. Anti-VEGFR antibodies are also described in U.S. application Ser. No. 09/976,787 (Zhu et al.). U.S. Pat. No. 6,177,401 (Ullrich et al.), U.S. Pat. No. 5,712,395 (App et al.), and U.S. Pat. No. 5,981,569 (App et al.) describe VEGFR antagonists that are organic molecules. In addition, bi-specific antibodies (BsAbs), which are antibodies that have two different antigen-binding specificities or sites, directed to VEGFR-2 and VEGFR-1 are known. See, e.g., U.S. application Ser. No. 09/865,198 (Zhu); 60/301,299 (Zhu).

Furthermore, assays for the determination of VEGFR antagonists are well known in the art, and alternate antagonists suitable for use in the present invention can be readily identified. The VEGFR antagonists of the present invention inhibit the tyrosine kinase activity of VEGFR, which generally involves phosphorylation events. Accordingly, phosphorylation assays are useful in determining VEGFR antagonists in the context of the present invention. Some assays for tyrosine kinase activity are described in Panek et al., J. Pharmacol. Exp. Thera., 283: 1433-44 (1997) and Batley et al., Life Sci., 62: 143-50 (1998). In addition, methods specific for detection of VEGFR expression can be utilized.

In another embodiment of the present invention, the VEGFR antagonist is a ligand, peptide or DNA. Any suitable ligand can be used that does not result in activation of the VEGFR, including mutants of the natural ligands VEGF and/or P1GF that do not bind the VEGFR. It should be appreciated that a skilled artisan can easily create VEGF and P1GF mutants that bind to the VEGFR, but that do not activate the receptor. Again, provided that the peptide or DNA binds to the VEGFR and does not active the VEGFR, any suitable peptide or DNA can be used in the context of the present invention.

Alternatively, the VEGFR antagonist can be a small molecule. Small molecules of the present invention are entities having carbon and hydrogen atoms, as well as heteroatoms, which include, but are not limited to, nitrogen, sulfur, oxygen, and phosphorus. Atoms in a small molecule are linked together via covalent and ionic bonds; the former is typical for small organic compounds, e.g., small molecule tyrosine kinase inhibitors, and the latter is typical of small inorganic compounds. The arrangement of atoms in a small organic molecule can represent a chain, e.g., a carbon-carbon chain or carbon-heteroatom chain, or ring containing carbon atoms, e.g., benzene, or a combination of carbon and heteroatoms, i.e., heterocycles, for example, a pyrimidine or quinazoline. A combination of one or more chains in a small organic molecule attached to a ring system constitutes a substituted ring system and fusion of two rings constitutes a fused policyclic system, which can be referred to as simply a policyclic system. Small molecules include both compounds found in nature, such as hormones, neurotransmitters, nucleotides, amino acids, sugars, lipids and their derivatives, and those compounds made synthetically, either by traditional organic synthesis, bio-mediated synthesis, or a combination thereof. See, e.g., Ganesan, Drug Discov. Today, 7(1): 47-55 (January 2002); Lou, Drug Discov. Today, 6(24): 1288-1294 (December 2001). Furthermore, small molecules include, for example, lipids and polymers of polysaccharides, as well as derivatives thereof, such as, e.g., lipopolysaccharides. Again, any suitable small molecule that prevents activation of the VEGFR can be used in the context of the present invention.

In one aspect of the invention, the VEGFR antagonists can be fused to additional amino acid residues, such as a peptide tag, to facilitate isolation or purification or a signal sequence to promote secretion or membrane transport in any particular host in which the antagonist is expressed.

The VEGFR antagonist of the present invention can be produced by any suitable method. For example, VEGFR antagonist antibodies, and particularly monoclonal antibodies, can be produced by methods known in the art. These methods include the immunological method described by Kohler and Milstein, Nature 256: 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA method described by Huse et al. in Science, 246: 1275-1281 (1989). Thus creating suitable hybridomas that express antibodies that are VEGFR antagonists. Specific hybridomas that produce VEGFR antibodies antagonists are exemplified.

Alternatively, DNA encoding the VEGFR antagonist can be cloned into a vector of an appropriate expression system. For example, HCMV vectors designed to express either human light chains of human heavy chains in mammalian cells can be utilized to express antibodies of the present invention. (See, e.g., U.S. Pat. No. 5,840,299; Maeda, et al., Hum. Antibod. Hybridomas, 2: 124-134 (1991)). Such vectors can contain a promoter and enhancer for high level transcription of the constructs, e.g., the human cytomegalovirus (CMV), replication origins and selectable markers functional in mammalian cells and E. coli.

A selectable marker is a gene that encodes a protein necessary for the survival or growth of transformed host cells grown in a selective culture medium. Typical selectable markers encode proteins that (a) confer resistance to antibiotics or other toxins, e.g. ampicillin, neomycin, methotrexate, or tetracycline, (b) complement auxotrophic deficiencies, or (c) supply critical nutrients not available from complex media, e.g. the gene encoding D-alanine racemase for Bacilli. A particularly useful selectable marker confers resistance to methotrexate. For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub & Chasin, Proc. Natl. Acad. Sci. USA, 77: 4216 (1980). The transformed cells are then exposed to increased levels of methotrexate. This leads to the synthesis of multiple copies of the DHFR gene, and, concomitantly, multiple copies of other DNA having the expression vectors, such as the DNA encoding the VEGFR antagonist.

Preferred host cells for transformation of vectors and expression of VEGFR antagonists of the present invention are mammalian cells, e.g., COS-7 cells, chinese hamster ovary (CHO) cells, and cell lines of lymphoid origin such as lymphoma, myeloma, or hybridoma cells. Other eukaryotic host, such as yeasts, can be alternatively used. The transformed host cells are cultured by methods known in the art in a liquid medium containing assimilable sources of carbon (carbohydrates such as glucose or lactose), nitrogen (amino acids, peptides, proteins or their degradation products such as peptones, ammonium salts or the like), and inorganic salts (sulfates, phosphates and/or carbonates of sodium, potassium, magnesium and calcium). The medium furthermore contains, for example, growth-promoting substances, such as trace elements, for example iron, zinc, manganese and the like.

Where it is desired to express a gene construct in yeast, a suitable selection gene for use in yeast is the trp1 gene present in the yeast plasmid YRp7. Stinchcomb et al. Nature, 282: 39 (1979); Kingsman et al., Gene, 7: 141 (1979). The trp1 gene provides a selection marker for a mutant strain of yeast lacking the ability to grow in tryptophan, for example, ATCC No. 44076 or PEP4-1. Jones, Genetics, 85: 12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

Also alternatively, the DNA encoding the VEGFR antagonist can be cloned into vectors derived from viruses such as adenovirus, adeno-associated virus, herpesvirus, retrovirus or lentivirus. Gene expression is controlled by inducible or uninducible regulatory sequences. The vector can also include origin of replication to ensure maintenance of vector, one or more selectable markers, leader sequences useful for directing secretion of translated polypeptide into the periplasmic space or extra-cellular medium of host cell.

Other sources of the VEGFR antagonists, and especially the VEGFR antagonist VEGFR-1, are receptors bound to the surface of cells. For example, the cells to which the VEGFR-1 is bound can be cells that naturally express the VEGFR-1 receptor, such as endothelial cells. Alternatively, the cell to which the full length or truncated VEGFR-1 is bound can be a cell into which the DNA encoding the VEGFR-1 has been transfected, such as 3T3 cells. These cells can be isolated from the mammal in accordance with methods known in the art.

In embodiments where the VEGFR antagonist is a peptide or DNA, the VEGFR antagonist of the invention can alternatively be prepared using standard solid phase (or solution phase) peptide synthesis methods, as is known in the art. In addition, the DNA can be synthesized using commercially available oligonucleotide synthesis instrumentation and produced recombinantly using standard recombinant production systems. Production using solid phase peptide synthesis is necessitated if non-gene-encoded peptides are to be included.

The present VEGFR antagonists can be administered for prophylactic and/or therapeutic treatments. In therapeutic applications, compositions are administered to a patient already suffering from ischemia or to a patient currently suffering as a result of an inflammatory disease. A “therapeutically effective dose” of the pharmaceutical composition, therefore, means an amount sufficient to stop, reverse or reduce the progression of the ischemia or inflammatory disease being treated. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. In prophylactic applications, compositions containing the present VEGFR antagonists are administered to either a patient not presently suffering from ischemia, but suffering from atherosclerosis or a patient having another inflammatory disease, but not currently suffering from the effects of it. A “therapeutically effective dose” of the pharmaceutical composition, therefore, means an amount sufficient to stop, reverse or reduce the progression of the atherosclerosis or other inflammatory disease being treated. In this use, again the precise amounts again depend upon the patient's state of health and general level of immunity, as well as dosing schedules, which are described previously.

The identification of those mammals that have atherosclerosis or other inflammatory disease is well within the ability and knowledge of one skilled in the art. For example, human individuals who are either suffering from clinically significant atherosclerosis (or other inflammatory disease) or who are at risk of developing clinically significant atherosclerosis (or other inflammatory disease) are suitable for administration of the present VEGFR antagonist. A clinician skilled in the art can readily determine, for example, by the use of clinical tests, physical examination and medical/family history if an individual is a patient has atherosclerosis or another inflammatory disease. Inflammatory diseases are numerous and well known in the art. Examples of inflammatory diseases include, but are in no way limited to, rheumatoid arthritis (RA), insulin-dependent diabetes mellitus, multiple sclerosis, myasthenia gravis, Crohn's disease, autoimmune nephritis, primary biliary cirrhosis, psoriasis, acute pancreatitis, allograph rejection, allergic inflammation, inflammatory bowel disease, septic shock, osteoporosis, osteoarthritis, and cognitive deficits induced by neuronal inflammation.

It should be appreciated that the VEGFR antagonist can be administered to any mammal. Specifically, the VEGFR antagonist of the present invention can be administered to a human. It is understood that the VEGFR antagonists of the invention, where used in the mammal for the purpose of prophylaxis or treatment, will be administered in the form of a composition additionally having a pharmaceutically acceptable carrier. For the purposes of this invention, the VEGFR antagonist can also be administered by various routes, e.g., orally, rectally, topically, or parenterally, by injection or infusion, in the form of a pharmaceutical composition.

Suitable pharmaceutically acceptable carriers include, for example, one or more of water, saline, phosphate buffered saline, dextrose, glycerol, ethanol and the like, as well as combinations thereof. Pharmaceutically acceptable carriers can further have minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The compositions of this invention can be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application.

Such compositions are prepared in a manner well known in the pharmaceutical art. In making the composition the active ingredient will usually be mixed with a carrier, or diluted by a carrier, and/or enclosed within a carrier, which can, for example, be in the form of a capsule, sachet, paper or other container. When the carrier serves as a diluent, it can be a solid, semi-solid, or liquid material, which acts as a vehicle, excipient or medium for the active ingredient. Thus, the composition can be in the form of tablets, lozenges, sachets, cachets, elixirs, suspensions, aerosols (as a solid or in a liquid medium), ointments containing for example up to 10% by weight of the active compound, soft and hard gelatin capsules, suppositories, injection solutions, suspensions, sterile packaged powders and as a topical patch.

Alternatively, the VEGFR antagonist can also be administered as a DNA encoding such VEGFR antagonist. Examples of suitable expression vector constructs are known in the art and have been described previously. Methods of DNA preparation also are known in the art and have been described previously.

In another aspect of the invention, the VEGFR antagonist can be chemically or biosynthetically linked to one or more anti-atherosclerotic agents or anti-inflammatory agents. The VEGFR antagonist can also be administered in combination with any other method of treatment of atherosclerosis or any other method of treatment of inflammatory disease known in the art, examples of which have been described previously. In addition, the VEGFR antagonist can be administered in combination with one or more suitable adjuvants, such as, e.g., cytokines or other immune stimulators.

Anti-inflammatory drugs include NSAIDs that exert anti-inflammatory, analgesic and antipyretic activity. These include salicylates such as aspirin, sodium salicylate, choline salicylate, salicylsalicylic acid, diflunisal, aloxiprine, lysine-acetyl salicylate, benorilate, calciumcarcasalate, and salsalate; indoleacetic acids such as indomethacin and proglumethacin; aryl-acetic acids such as bufexamac, diclofenac, tolmetin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as nifluminic acid, mefanamic acid, and meclofenamate; oxicams such as piroxicam and tenoxicam; naphthaleneacetic acids such as naproxen; and gold salts such as sodium aurothiopropanolsulphonate and auranofin. Adrenal corticosteroids are alternatives to NSAIDs for treating inflammatory diseases. These steroids include hydrocortisone, prednisolone, 6 alpha-methylprednisolone, triamcinolone, dexamethasone and betaroethasone. In addition, the anti-inflammatory effect of glucocorticoids has been well documented.

The invention further contemplates VEGFR antagonists of the invention to which target or reporter moieties are linked. Target moieties are first members of binding pairs. Anti-atherosclerotic agents, for example, are conjugated to second members of such pairs and are thereby directed to the site where the VEGFR antagonist is bound. A common example of such a binding pair is avidin and biotin. In a preferred embodiment, biotin is conjugated to a VEGFR antagonist of the invention, and thereby provides a target for an anti-atherosclerotic agent or other moiety (e.g., an anti-inflammatory agent), which is conjugated to avidin or streptavidin. Alternatively, biotin or another such moiety is linked to a VEGFR antagonist of the invention and used as a reporter, for example in a diagnostic system where a detectable signal-producing agent is conjugated to avidin or streptavidin.

Accordingly, the present VEGFR antagonists thus can be used in vivo and in vitro for investigative, diagnostic, prophylactic, or treatment methods, which are well known in the art. Of course, it is to be understood and expected that variations in the principles of invention herein disclosed can be made by one skilled in the art and it is intended that such modifications are to be included within the scope of the present invention.

The examples that follow further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook, J. et al., (1989) Molecular Cloning: A Laboratory Manual, 2^{nd} ed., Cold Spring Harbor Laboratory Press. All references mentioned herein are incorporated in their entirety.

### EXAMPLES

Anti-VEGFR-1 Antibody

The rat anti-VEGFR-1 monoclonal antibody, MF-1, was developed through a standard hybridoma technique. Eight weeks old rats were primed intraperitoneally (i.p.) with 100 μg of VEGFR-1 Fc (constant region) recombinant protein (R&D Systems, Minneapolis, Minn.) mixed with complete Freunds adjuvant. Then, the rats were boosted three times prior to fusion with the same protein mixed with incomplete Freunds adjuvant. Hybridoma cells were generated by fusing myeloma cells P3x63Ag8.653 with spleen cells and bone marrow cells from immunized rats. Anti-VEGFR-1 specific clones were selected using VEGFR-1 alkaline phosphatase (AP) recombinant protein in ELISA-based binding and blocking assays. Positive clones were subcloned by limiting dilution.

Anti-VEGFR-1 monoclonal antibodies (mAbs) from hybridomas were obtained via continuous feed fermentation in serum-free medium. The mAbs were purified from serum-free conditioned media by affinity chromatography using Gamma-bind protein G-Sepharose. The mAbs used in in vivo studies were tested for endotoxin using the PYROGENT PLUS® Limulus Amebocyte Lysate kit (Bio Whittaker, Walkersville, Md.). All antibody preparations used in animal studies contained ≦1.25 EU/ml of endotoxin. Anti-VEGFR-1 polyclonal antibodies were generated from recombinant VEGFR-1 AP protein immunized rabbit and purified by Gamma-bind protein G column (Amersham Pharmacia Biotech, Uppsala, Sweden).

VEGFR-1 Binding Assay and VEGF/P1GF Blocking Assays

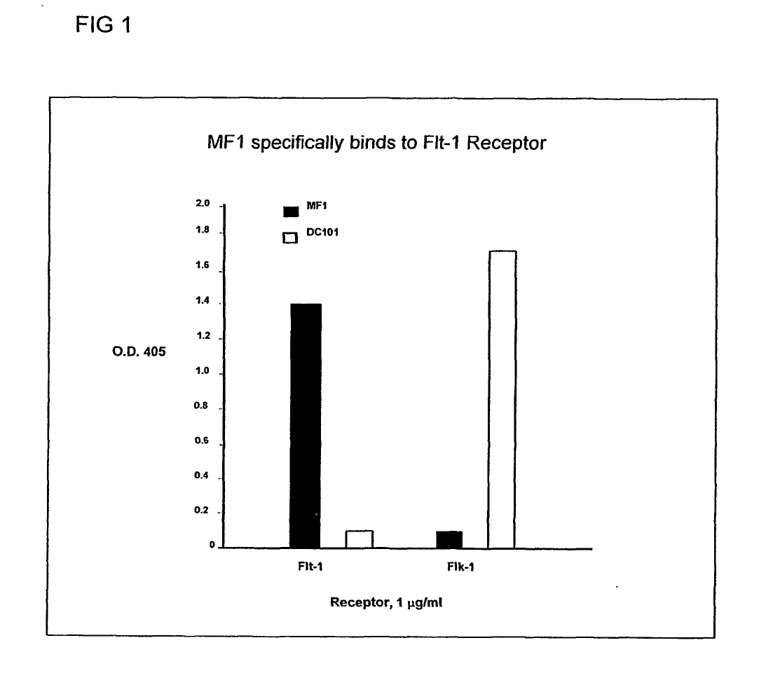

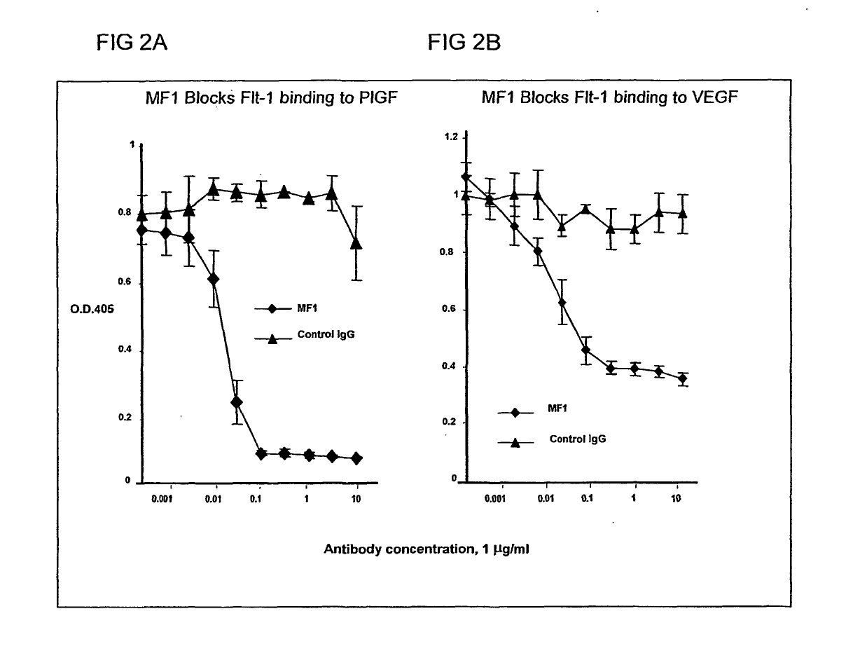

The immunochemical properties of anti-VEGFR-1 mAbs were characterized in ELISA-based binding and blocking assays. FIG. 1 shows the results from the binding assays and FIG. 2 shows the results from the blocking assays.

Binding assays were performed by coating 96-well microtiter plates (Falcon Flexible plate, Becton Dickinson, Bedford, Mass.) with 50 ng/well VEGFR-1 AP or VEGFR-2 AP protein overnight at 4° C. Wells were blocked by adding 200 μl of phosphate-buffered saline containing 5% bovine serum, 0.05% Tween 20 (blocking buffer) and incubating for 2 hrs at room temperature (RT). Wells were then washed (5×) and incubated for 1 hr at RT with various concentrations of mAbs at 50 μl diluted in blocking buffer. Wells were again washed (5×) and incubated with 50 μl of goat anti-rat IgG-HRP (BioSource International, Camarillo, Calif.) for 1 hr at RT. Wells were washed (5×) for a final time and then incubated with 50 μl of 3,3′,5,5′-tetra-methylbenzidine (TMB) substrate (Kirkegaard and Perry Lab Inc., Gaithersburg, Md.) for 15 mins at RT. The reaction was stopped by adding 50 μl of 1 M Phosphoric Acid (H₃PO₄) and wells read at 405 nm on a microtiter plate reader. As shown in FIG. 1, MF-1 specifically binds to VEGFR-1 and does not bind to VEGFR-2.

For VEGFR-1/VEGF or P1GF blocking assays, wells were coated with 100 ng of VEGF or P1GF (R & D Systems, Minneapolis, Minn.) overnight at 4° C. Wells are blocked as described above and then incubated for 1 hr at RT with 100 ng of VEGFR-1 AP that had been preincubated for 1 hr with various concentrations of mAb. Wells were washed and incubated with p-nitrophenyl phosphate (PNPP, Sigma, St. Louis, Mo.). Color was developed for 30 mins at RT and was then read at 405 nm on a microtiter plate reader. As shown in FIG. 2A, MF-1 blocks VEGFR-1 binding to P1GF and in FIG. 2B, MF-1 blocks binding of VEGFR-1 to VEGF.

Inflammatory Disease

Rheumatoid arthritis (RA) is a prime example of an inflammatory disease, and is thus the inflammatory disease exemplified. To study the efficacy of MF-1 treatment during RA, an auto-immune mouse model of collagen II (CII)-induced arthritis (CIA) was used, since this model has proven to resemble in many ways RA development in humans.

Eight to ten-week-old DBA/1JO1a mice were purchased from Harlan (Horst, The Netherlands). For all experiments, the male to female ratio was kept between 0.8 and 1.3 in each treatment group. For induction of CIA, native chicken collagen type II (Elastin Products Company, Owensville, USA) was dissolved in 0.05M acetic acid at 2 mg/ml by stirring overnight at 6° C. and emulsified in an equal volume of CFA (Difco, Detroit, USA), with added Mycobacterium butyricum (0.5 mg/ml). Mice were immunized on day 0 by single subcutaneous injection of 100 μl of the collagen-emulsion in complete Freund's adjuvant (CFA) at the base of the tail. At day 21, mice were re-injected with 100 μl of emulsion containing 0.5 mg/ml of native chicken collagen type II in incomplete Freund's adjuvant (IFA; Difco, Detroit, USA). From day 10 on, mice were weighed and clinically examined daily for the presence of arthritis. Disease severity was recorded according to a scoring system for each paw.

Treatment with rat anti-mouse VEGFR-1 antibodies (MF-1, described above) was initiated at day 10 (before the onset of disease) by intraperitoneal injection of 750 μg/mouse and continued every three days until sacrifice. Control mice were treated at the same days with an equal dose of rat immunoglobulins (IgG; Sigma, St. Louis, USA). At day 45, mice were sacrificed, fore and hind paws were isolated and ankles were separated from the hind paws. Paws and ankles were fixed in 10% formol, decalcified in formic acid (31% (v/v) formic acid and 13% (m/v) sodium citrate) and embedded in paraffin. Ten μn sections were prepared and stained with hematoxilin-eosin for histological evaluation. Two investigators, unaware of the treatment regimen, determined histological score according to a standard scoring protocol.

MF-1 treated mice had a normal weight gain, comparable to control mice, indicating that MF-1 was not toxic at the dose used in this study (750 μg every three days). In addition, analysis of the spleens, the livers, the kidneys, the lungs and hearts of treated mice did not reveal any abnormalities.

### Example 1

The present example investigates the role of a VEGFR antagonist in early atherosclerotic plaque development. Specifically, the present example investigates the effect of treatment with anti-VEGFR-1 antibodies in atherosclerosis-prone apolipoprotein E-deficient mice (apoE−/− mice). These apoE-deficient mice were fed a high cholesterol diet starting at 5 weeks of age, at which time the mice had no signs of atherosclerosis. At 5 weeks, the mice were injected with 500 μg anti-VEGFR-1 antibody (rat anti-mouse antibody MF-1 described above) or 500 μg control rat immunoglobulins (IgGs) (Sigma, Borneum, Belgium) three times per week for a period of 5 weeks. The aortas were perfused, embedded in paraffin and 7 μm sections were stained with hematoxylin-eosin for morphometric analysis.

Mice in both treatment groups had a normal weight gain (21±1 g for anti-VEGFR-1 treated mice versus 21±1 in control IgG treated mice; n=20, p=NS) and developed fatty livers (liver weight: 1.72±0.11 g for anti-VEGFR-1 treated mice versus 1.69±0.08 g in control IgG treated mice; n=20, p=NS). Treatment with anti-VEGFR-1 did not affect total plasma cholesterol levels (1600±90 mg/dl for anti-VEGFR-1 treated mice versus 1640±140 mg/dl in control IgG treated mice; n=20, p=NS).

All mice developed macrophage-rich lesions but anti-VEGFR-1 treatment resulted in a 2.5-fold reduction in lesion size at the aortic root (cross-sectional plaque area: 12000±1800 μm² in anti-VEGFR-1 treated mice versus 25000±2200 μm² in control IgG treated mice; n=20, p=0.0001). In the thoracic and abdominal aorta, anti-VEGFR-1 antibodies also reduced the size of the lesions (cross-sectional plaque area: 3500±190 μm² in anti-VEGFR-1 treated mice versus 4400±250 m² in control IgG treated mice; n=10, p=0.009).

Lesions of anti-VEGFR-1 treated mice contained significantly less macrophages (area occupied by the macrophage-marker Mac-3: 5200±600 μm² in anti-VEGFR-1 treated mice versus 12000±1600 μm² in control IgG treated mice; n=10, p=0.01). The endothelium was comparably activated in both treatment groups, as indicated by the comparable upregulation of VCAM-1 expression (number of RNA copies/100 copies HPRT: from 1380±320 in non-atherosclerotic aorta to 4080±430 in atherosclerotic aorta of anti-VEGFR-1 treated mice versus from 1260±130 in non-atherosclerotic aorta to 4580±790 in atherosclerotic aorta of control IgG treated mice; n=4, p=NS).

The number of circulating monocytes was significantly down-regulated by anti-VEGFR-1 treatment, as determined by FACS analysis (number of circulating monocytes (CD11b+/Gr-1low)/μl blood: 160±17 in anti-VEGFR-1 treated mice versus 270±45 in control-IgG treated mice, n=12, p=0.03) without affecting circulating lymphocyte levels (number of circulating lymphocytes (CD19+ or CD3ε+)/μl blood: 1100±130 in anti-VEGFR-1 treated mice versus 1150±290 in control-IgG treated mice, n=12, p=NS).

In addition to monocytes, the circulating levels of granulocytes were also down regulated (number of circulating granulocytes (CD11b+/Gr-1high)/μl blood: 980±190 in anti-VEGFR-1 treated mice versus 1670±320 in control-IgG treated mice, n=12, p=0.08), indicating that anti-VEGFR-1 directly acted on inflammatory cells.

Thus, the present example demonstrates treatment of atherosclerosis with a VEGFR antagonist. Furthermore, the present example demonstrates a decrease in inflammatory-related cells such as macrophages, monocytes and granulocytes.

### Example 2

The present example investigates the role of a VEGFR antagonist in advanced atherosclerotic plaque development. Specifically, the present example investigates the effect of treatment with anti-VEGFR-1 antibodies in atherosclerosis-prone apolipoprotein E-deficient mice (apoE−/− mice). These apoE-deficient mice were fed a high cholesterol diet starting at 5 weeks of age and at 20 weeks of age, the mice were injected with 500 μg anti-VEGFR-1 antibody (rat anti-mouse antibody MF-1 described above) or 500 μg control rat immunoglobulins (IgGs) (Sigma, Borneum, Belgium) three times per week for a period of 5 weeks (until 25 weeks of age). The aortas were perfused, embedded in paraffin and 7 μm sections were stained with hematoxylin-eosin for morphometric analysis.

Mice in both treatment groups had a normal weight gain (29±1 g for anti-VEGFR-1 treated mice versus 28±1 in control IgG treated mice; n=8, p=NS) and developed fatty livers (liver weight: 2.14±0.33 g for anti-VEGFR-1 treated mice versus 1.81±0.10 g in control IgG treated mice; n=8, p=NS). Treatment with anti-VEGFR-1 did not affect total plasma cholesterol levels (1640±90 mg/dl for anti-VEGFR-1 treated mice versus 1500±110 mg/dl in control IgG treated mice; n=8, p=NS).

All mice developed advanced atherosclerotic lesions with large necrotic cores and fibrous caps but anti-VEGFR-1 treatment resulted in a 22% reduction in lesion size at the aortic root (cross-sectional plaque area: 210000±22000 μm² in anti-VEGFR-1 treated mice versus 270000±20000 μm² in control IgG treated mice; n=8, p=0.03). As in early lesions, the area occupied by macrophages was almost two-fold reduced in lesions of ant-VEGFR-1 treated mice (37000±9000 μm² versus 60000±8000 μm² in control IgG treated littermates, n=8, p=0.03).

Treatment with anti-VEGFR-1 did not affect collagen deposition in the lesions (area occupied by the collagen dye Sirius red: 95000±15000 μm² in anti-VEGFR-1 treated mice versus 115000±15000 μm² in control IgG treated littermates, n=8, p—NS).

### Example 3

The present example further investigates the role of a VEGFR antagonist in atherosclerotic plaque development. Specifically, the present example investigates the effect of treatment with anti-VEGFR-1 antibodies in atherosclerosis-prone apoE−/− mice. ApoE-deficient mice of the C57BL/6 genetic background were fed with a standard chow diet and were treated chronically for 4 weeks with anti-VEGFR-1 antibody (1 mg/dose i.p. every 3 days). Mice were started on this treatment at either 6 or 10 weeks of age, and subsequently sacrificed for determination of atherosclerotic lesion areas in the aortic root at 10 or 14 weeks of age, respectively. Hearts with the attached aortic roots were fixed in 10% buffered formalin, embedded in gelatin, frozen in OCT, and cut into 10 μm sections. Sections were stained with oil red 0, and the mean lesion per section was measured over the first 500 μM of the aortic sinus.

For the mice treated between 6 and 10 weeks of age, the median lesion area was reduced by 52% in the treated mice compared to the control group (30,700 μm² vs. 63,600 μm²; n=5). For the mice treated between 10 and 14 weeks of age, the median lesion area was reduced by 47% in the treated mice compared to the control group (75,500 μm² vs. 143,800 μm²; n=5). These alterations were observed without significant changes in total plasma cholesterol levels due to the antibody treatment, in fact, total cholesterol levels were higher in the mice treated from 10-14 weeks of age. Thus, even though cholesterol levels were elevated in this group, there was decreased atherosclerosis.

Accordingly, the present example demonstrates treatment of atherosclerosis with a VEGFR antagonist.

### Example 4

The present example investigates the effect of a VEGFR antagonist on the development and progression of an inflammatory disease. To study the efficacy of anti-VEGFR1 antibody (MF-1)-treatment during RA, an autoimmune mouse model of collagen II (CII)-induced arthritis (CIA) was used, since this model has proven to resemble in many ways RA development in humans. Groups of 9 mice were evaluated for body weight (g±SEM). MF-1 treated mice had a normal weight gain, comparable to control mice (Table 1, which is a body weight analysis in MF-1 and control treated DBA/1 mice), indicating that MF-1 was not toxic at the dose used in this study (750 μg every three days). In addition, analysis of the spleens, the livers, the kidneys, the lungs and hearts of treated mice did not reveal any abnormalities.

TABLE 1

| | Control | MF-1 |
|---|---|---|
| Days After Immunization | (g ± SEM) | (g ± SEM) |
| 0 | 21.0 ± 1.1 | 20.5 ± 0.9* |
| 10 | 22.4 ± 1.0 | 22.5 ± 0.9* |
| 22 | 23.0 ± 1.1 | 23.4 ± 0.9* |
| 34 | 23.6 ± 0.8 | 24.2 ± 0.8* |
| 45 | 24.1 ± 0.8 | 26.2 ± 0.9* |

*P = NS by unpaired Student's t-test.

Mice were clinically treated on a daily basis for the presence of arthritis in their paws and ankles. By the end of the study, treatment with MF-1 reduced the cumulative incidence of arthritis by 25%. Comparison of the clinical scores (based on paw swelling, erythema and ankylosis) reveled that MF-1 treatment gradually reduced clinical severity which became statistically significant as early as 7 days after clinical onset. At the end of the follow-up period, the mean clinical score in the MF-1-treated group was three-fold lower than in the control-treated mice (2.1±1.0 in MF-1 treated mice versus 6.4±1.4 in control-treated mice, n—10, P<0.05).

Susceptibility to CIA has been found to be associated with high anti-CII antibody responses. Therefore, the humoral response was assessed by measuring anti-CII antibodies (total IgG) in the sera obtained from the mice at the time of sacrifice (day 45 post immunization). Serum levels of anti-CII antibodies were not affected by MF-1 treatment (total anti-CII IgG in μg/ml: 930±200 in MF-1-treated mice versus 970±170 in control-treated mice; n=9, P=NS).

Clinical improvement by MF-1 treatment was confirmed by histological analysis of the fore- and hind paws and the ankles, according to standard protocols. At the time of sacrifice, all mice (10 out of 10) in the control-treated group had histological signs of arthritis in at least one paw, while 2 out of 9 MF-1 treated mice had no signs of disease. Joints of all paws and ankles were individually scored for the presence and degree of synovial hyperplasia (H), inflammation (1) and pannus formation (P) and the additive score (calculated by adding up the scores of all paws and ankles) was determined for every mouse. The mean additive histological score for the three parameters (H, I and P) was significantly reduced in the MF-1-treated group as compared to the control-treated group (H: 6.6±1.2 in control-treated versus 2.4±0.9 in MF-1 treated mice; I: 6.1±1.3 in control-treated versus 2.1±0.9 in MF-1 treated mice; P: 4.8±1.1 in control-treated versus 1.4+0.7 in MF-1 treated mice).

To evaluate whether MF-1 treatment directly acted on inflammatory cells, the number of circulating mononuclear and polymorphonuclear cells was determined by FACS analysis. MF-1 treatment reduced both the levels of monocytes and granulocytes (number of cells/μl peripheral blood: monocytes (CD11b+/Gr-1low): 280±65 in MF-1-treated mice versus 640±200 in control-treated mice; n=12, P=0.09; granulocytes (CD11b+, Gr-1high): 980±90 in MF-1-treated mice versus 1650±250 in control-treated mice; n—12, P=0.009), whereas lymphocyte levels were not affected by MF-1 (number of lymphocytes (CD19+ or CD3ε+)/μl peripheral blood: 1400±220 in MF-1-treated mice versus 1200±240 in control-treated mice; n—12, P=NS).

To further evaluate inflammation, immunohistochemical analysis was performed on the most severely affected fore paw for every mouse. Inflammation was studied by measuring the area (expressed as density in %) positive for CD45, a pan-leukocytic marker. Mean density was more than two-fold reduced in the MF-1 treatment group, indicating that MF-1 decreased infiltration of inflammatory cells (7.5+1.8% in MF-1-treated mice versus 20.3±2.9% in control-treated mice, n=8; P<0.05). Staining of serial sections with antibodies against macrophages (Mac-3), revealed that the majority of inflammatory cells were from the macrophage lineage (Mac-3 density: 7.7±1.6% in MF-1-treated mice versus 16.0±1.9% in control-treated mice, n=8; P<0.05).

Degradation of the cartilage covering the bone, supposedly by MMPs, is an important feature of joint destruction during RA. Depletion of cartilage proteoglycans is one of the first signs of cartilage destruction, followed by degradation of collagen type II. To analyze cartilage destruction, sections of ankles were stained with safranin O for proteoglycans. Safranin O-positive area was measured and normalized for the length of the bone covered by cartilage at two different locations (talus/calcaneus and tibia/talus interface). Treatment with MF-1 significantly decreased cartilage destruction as evidenced by the increased area occupied by safranin 0 at both locations (area in μm² per μm at the talus/calcaneus interface: 90.5±4.3 in the MF-1-treated group versus 64.2±6.6 in the control-treated group; area in μm² per μm at the tibia/talus interface: 79.2±2.9 in the MF-1-treated group versus 57.3±5.1 in the control-treated group, n=8, P<0.05).

The importance of new capillary growth in the development of synovitis has long been established. These new vessels deliver oxygen, nutrients and inflammatory cells to the invasive pannus. To determine whether MF-1 treatment affected vessel growth, the number of capillaries was determined in CD31-stained sections of the most severely affected fore paws for every mouse and expressed as capillary density. Mean capillary density was significantly reduced in MF-1-treated mice, indicating that MF-1 decreased synovial neovascularization (number of capillaries/mm²: 340±64 in MF-1-treated mice versus 525±25 in control-treated mice, n=8; P<0.05).

1. A method of treating atherosclerosis in a mammal comprising administering to the mammal a therapeutically effective amount of a vascular endothelial growth factor receptor (VEGFR) antagonist.

2. A method of treating inflammatory disease in a mammal comprising administering to the mammal a therapeutically effective amount of a vascular endothelial growth factor receptor (VEGFR) antagonist.

3. The method of claim 2, wherein the inflammatory disease is arthritis.

4. The method of claim 1, wherein the arthritis is rheumatoid arthritis (RA).

5. The method of claim 1, wherein the mammal is a human.

6. The method of claim 1, wherein the VEGFR is fms-like tyrosine kinase receptor (flt-1) or VEGFR-1.

7. The method of claim 1, wherein the VEGFR is kinase insert domain-containing receptor/fetal liver kinase (KDR/flk-1) or VEGFR-2.

8. The method of claim 1, wherein the VEGFR antagonist inhibits tyrosine kinase activity.

9. The method of claim 1, wherein the VEGFR antagonist is an antibody.

10. The method of claim 9, wherein the antibody comprises a constant region of a human antibody.

11. The method of claim 10, wherein the antibody is a chimeric antibody comprising a variable region of a mouse antibody.

12. The method of claim 10, wherein the antibody is a humanized antibody comprising complementarity-determining regions (CDRs) of a mouse antibody and framework regions of a human antibody.

13. The method of claim 10, wherein the antibody is a human antibody comprising a variable region of a human antibody.

14. The method of claim 1, wherein the VEGFR antagonist prevents VEGF and P1GF from binding to the receptor.

15. The method of any of claim 1, wherein the VEGFR antagonist inhibits binding of ATP to VEGFR.

16. The method of claim 15, wherein the VEGFR antagonist is a small molecule.

17. The method of claim 1, wherein the method further comprises administering an adjuvant.
